(19) United States
(12) Patent Application Publication
(10) Pub. No.: US 2024/0000955 A1
(43) Pub. Date: Jan. 4, 2024

(54) SOLUBLE POLYELECTROLYITIC COMPLEXES OF POLYMERIC BIGUANIDINE MICROBIOCIDES WITH GLYCOSAMINOGLYCANS

(71) Applicant: Altergon S.A., Lugano (CH)

(72) Inventors: Chiara Schiraldi, Lugano (CH); Mario De Rosa, Lugano (CH); Tiziano Fossati, Lugano (CH)

(73) Assignee: Altergon S.A., Lugano (CH)

(21) Appl. No.: 18/254,864

(22) PCT Filed: Nov. 29, 2021
(86) PCT No.: PCT/IB2021/061053

§ 371 Date: May 29, 2023

(30) Foreign Application Priority Data
Dec. 1, 2020 (IT) 102020000029288

(51) Int. Cl. A61K 47/61

(52) U.S. Cl. CPC A61K 47/61

(57) ABSTRACT

Disclosed are soluble polyelectrolyte complexes of polymeric biguanidine antimicrobial, such as polyhexamethylene biguanide or polyaminopropyl biguanide, with glycosaminoglycans, such as hyaluronic acid, chondroitin or chondroitin sulphate and derivatives thereof, and their use as active ingredients of pharmaceutical compositions or medical devices.

The invention relates to soluble polyelectrolyte complexes of polymeric biguanidine microbiocides with glycosaminoglycans, and formulations containing them.

### PRIOR ART

Polyelectrolytes are polymers which have numerous ionisable functional groups on their structure. In solution, ionised polyelectrolytes can form complexes with oppositely charged polyelectrolytes; the behaviour of said complexes in solution is based on the stoichiometry between anionic polyelectrolyte equivalents and cationic polyelectrolyte equivalents.

Water-soluble polyelectrolyte complexes can be formed by mixing anionic and cationic polyelectrolytes in non-stoichiometric ratios, in practice involving a large excess of one to the other; however, when said ratio approximates to 1, insoluble polyelectrolyte complexes are obtained.

The cationic polyelectrolyte category includes polyhexamethylene biguanide antimicrobials, also called PHMBs, and polyaminopropyl biguanide, also called PAPBs, while GAGs are anionic polyelectrolytes.

PHMBs and PAPBs (collectively abbreviated to ABPs, i.e. Antimicrobial Biguanide Polymers), are synthetic polyelectrolytes with applications as antiseptics in the surgical and hygiene fields. PHMBs are used in the treatment of Acanthamoeba keratitis, as an ingredient of contact lens cleaning products, and in cosmetics, deodorants and veterinary products.

GAGs are natural anionic polyelectrolytes used in the pharmaceutical and cosmetic fields for their tissue-regenerating properties and rheological characteristics. Examples of GAGs are hyaluronic acid (HA), chondroitin (C) and chondroitin sulphate (CS).

The known formulations of biguanide antiseptic salts are not wholly satisfactory, and in particular do not allow delayed/controlled release forms, which are desirable in various applications. Formulations that combine the tissue-repair and wound-healing effects typical of GAGs with the known antibacterial and antiseptic properties of biguanide compounds would also be of interest.

### DESCRIPTION OF THE INVENTION

It has now been found that the interaction between ABPs and GAGs forms polyelectrolyte complexes whose characteristics depend on the stoichiometry with which the complex is formed. In particular, soluble polyelectrolyte complexes can only be obtained if the ratio in equivalents between GAG and ABP is greater than 10; this means that the polycation ABP must represent, in terms of equivalents, less than one-tenth of the positive charges that interact with the GAG, mainly consisting of monovalent alkali metal cations. Treatment of infected sites with said soluble polyelectrolyte complexes (abbreviated to GAG/ABP) is particularly advantageous due to the simultaneous, controlled, gradual and/or delayed release of ABP and GAG, synergising the antimicrobial action of ABP with the wound-healing and tissue-repair properties typical of GAG. ABP is released from GAG/ABP due to competition on the surface of the GAG by other cations present at the site of application.

In view of said characteristics, the GAGs/ABPs forming the object of the invention are particularly useful for the formulation of pharmaceutical compositions and medical devices.

The GAG is selected from HA, C and CS and mixtures thereof, hybrid cooperative complexes obtained as described in EP 2 614 090, and crosslinked forms as described in WO 2013/164782 A1.

The average molecular weight Mn of the polyelectrolytes broadly ranges between 2×10³ and 5×10⁶ Da.

Control of the ratio in equivalents between GAG and ABP enables the solubility of the GAG/ABP polyelectrolyte complex to be modulated.

The compounds according to the invention can easily be prepared in aqueous solution by mixing the GAG or a salt thereof with a salt of the ABP in the desired stoichiometric ratio.

At ratios between GAG equivalents and ABP equivalents >10, soluble polyelectrolyte complexes are obtained, while at smaller ratios, insoluble polyelectrolyte complexes are obtained.

The formation reaction of the GAG/ABP polyelectrolyte complex takes place immediately at room temperature. The complex can be isolated by filtration or centrifugation if insoluble, and by drying, freeze-drying or membrane processes if soluble.

The soluble GAG/ABP polyelectrolyte complexes according to the invention can be used in the form of eyedrops, gel, mouthwash, lotion, ointment, solution, medicated patch or any form suitable for topical administration.

Pharmaceutical compositions or medical devices containing soluble GAG/ABP polyelectrolyte complexes are a further object of the invention.

Said compositions are useful for the treatment of acne, and bladder, vaginal, eye and oral mucosa infections or inflammations.

The rheology and efficacy of formulations containing the soluble GAG/ABP polyelectrolyte complexes can be optimised by using rheology modifiers and other active ingredients or excipients.

In the formulations, the concentration of the soluble GAG/ABP polyelectrolyte complexes can range within wide limits, depending on the application in question. Broadly speaking, the concentration will range between 0.001 and 10% by weight of the total formulation.

The following examples describe the invention in more detail.

### Example 1—Formation of Polyelectrolyte Complexes of GAGs with PHMB

200 mL of the following solutions of GAG sodium salt are prepared at the concentration of 9.96 mEq/100 (sol. A), wherein the GAG is HA Mw 700 KDa or HA Mw 100 KDa or chondroitin 35 KDa, and a solution of PHMB hydrochloride Mw 2.2 KDa at the concentration of 45.77 mEq/100 mL (sol. B). Variable amounts of solution B are added to 10 mL of solution A under stirring, the volume is made up to 20 mL with distilled water, and stirring is continued for 4 h. If a precipitate forms the sample is centrifuged and the sediment is recovered by centrifugation and washed with 5 mL of water, stove-dried under vacuum and weighed. As shown in Table 1, a soluble polyelectrolyte complex is prevalently formed for GAG equivalent/PHMB equivalent ratio values >10, while the insoluble polyelectrolyte complex is the prevalent species with a ratio value <2; in the 2-10 range there is a balance between insoluble and soluble polyelectrolyte complex.

TABLE 1
Behaviour of GAG/PHMB system as a function of the stoichiometric
ratio GAG mEq/PHMB mEq (1.1, HA Mw 700 KDa; 1.2, HA
Mw 100 KDa; 1.3, chondroitin Mw 35 KDa). The data demonstrate
very similar behaviour on variation of the structural
characteristics of the GAG.

| | H₂O | PHMB | mEq GAG/ | Precipitate |
|---|---|---|---|---|
| B* mL | (mL) | (mEq) | mEq PHMB | (mg) |
| 3.84 | 6.16 | 1.76 | 0.56 | 590 |
| 1.52 | 8.46 | 0.70 | 1.42 | 296 |
| 0.77 | 9.23 | 0.35 | 2.81 | 98 |
| 0.38 | 9.62 | 0.18 | 5.72 | 40 |
| 0.22 | 9.78 | 0.10 | 10.00 | 22 |
| 0.11 | 9.89 | 0.05 | 19.91 | 2 |
| 0.05 | 9.95 | 0.03 | 39.82 | 0 |
| 3.84 | 6.16 | 1.76 | 0.56 | 530 |
| 1.52 | 8.46 | 0.70 | 1.42 | 300 |
| 0.77 | 9.23 | 0.35 | 2.81 | 95 |
| 0.38 | 9.62 | 0.18 | 5.72 | 48 |
| 0.22 | 9.78 | 0.10 | 10.00 | 10 |
| 0.11 | 9.89 | 0.05 | 19.91 | 1 |
| 0.05 | 9.95 | 0.03 | 39.82 | 0 |
| 3.84 | 6.16 | 1.76 | 0.56 | 570 |
| 1.52 | 8.46 | 0.70 | 1.42 | 280 |
| 0.77 | 9.23 | 0.35 | 2.81 | 91 |
| 0.38 | 9.62 | 0.18 | 5.72 | 33 |
| 0.22 | 9.78 | 0.10 | 10.00 | 11 |
| 0.11 | 9.89 | 0.05 | 19.91 | 0 |
| 0.05 | 9.95 | 0.03 | 39.82 | 0 |

1.1 - *solution B, pre-diluted to 10 mL with water, is added to 10 mL of GAG solution (HA Mw 700 KDa 0.996 mEq/10 ml).
1.2 - *solution B, pre-diluted to 10 mL with water, is added to 10 mL of GAG solution (HA Mw 100 KDa 0.996 mEq).
1.3 - *solution B, pre-diluted to 10 mL with water, is added to 10 mL of GAG solution (chondroitin Mw 35 KDa 0.996 mEq).

### Example 2—Slow Release of PHMB from the Soluble Polyelectrolyte Complex of Example 1.1 when the GAG mEq/PHMB mEq Ratio is 10

200 mL of the sample mEq HA 700 KDa/mEq PHMB equal to 10 of Example 1.1 is prepared, and 50 ml of the solution is transferred to a dialysis tube with a cut-off of 5 KDa and left to dialyse under stirring against 500 mL of saline solution. The absorption of the solution in the dialysis tube is determined at 240 nm over time. The dialysis kinetics of a solution containing the same concentration of PHMB (20 mL of PHMB solution in saline dialysed against 200 mL of saline) are monitored in parallel. Table 2 shows the absorption data at 240 nm, demonstrating that the dialysis balance in the case of PHMB alone is reached in about 12 h, whereas in the presence of HA, the balance is not yet reached after 24 h. This behaviour demonstrates that the soluble polyelectrolyte complex HA/PHMB behaves like a PHMB slow-release system, ensuring a gradual, continuous antimicrobial action over time.

Table 2—200 mL of polyelectrolyte complex HA/PHMB, wherein the mEq HA/mEq PHMB ratio is 10, is prepared as described in Example 1.1. 50 mL of solution is transferred to a dialysis tube with a cut-off of 5 KDa, and left to dialyse under stirring against 500 mL of saline solution. The absorption of the solution in the dialysis tube is determined at 240 nm over time. The dialysis kinetics of a solution containing the same amount of PHMB (20 mL of PHMB solution in saline dialysed against 200 mL of saline) are monitored in parallel. The absorption data demonstrate that the dialysis balance in the case of PHMB alone is reached in about 12 h, whereas in the presence of HA the balance is not yet reached after 24 h.

TABLE 2

| | 10 min | 1 h | 6 h | 12 h | 24 h |
|---|---|---|---|---|---|
| HA/PHMB* | 2.35 | 1.85 | 0.99 | 0.81 | 0.50 |
| PHMB | 2.28 | 1.33 | 0.40 | 0.21 | 0.21 |

*prepared as for mEq HA/mEq PHMB 10 of Table 1.1, but on a scale of 10 times higher.

### Example 3

Antimicrobial Activity Against Cutibacterium acnes of Various Soluble HA Mw 500 KDa/ABP Polyelectrolyte Complexes.

The Cutibacterium acnes strain (ATCC® 12827) was cultured anaerobically for 48 h in Schaedler Broth at 37° C. The culture broth obtained after incubation was diluted to the concentration of about 1.0-5.0×10⁸ CFU/ml. The assay was conducted anaerobically for 48 h at 37° C., and after incubation, the presence of bacterial growth was evaluated in each well. Different dilutions (0.01, 0.005, 0.001 and 0.0005) of solutions containing 0.1 mEq of PHMB or 0.1 mEq of PAPB or 1 mEq HA Mw 500 KDa/0.1 mEq PHMB or 1 mEq HA Mw 500 KDa/0.1 mEq PAPB in 10 mL were added to the various wells. Table 3 shows the results obtained. Up to the dilution of 0.001, both soluble polyelectrolyte complexes proved effective against Cutibacterium acnes 11827.

TABLE 3
Inhibition of the growth of Cutibacterium acnes
11827 in the presence of soluble polyelectrolyte
complexes HA Mw 500 KDa/PHMB and HA Mw 500 KDa/PAPB
wherein the ratio in HA/ABP equivalents is 10.

Dilution factors (%)

| Samples | 0.01 | 0.005 | 0.001 | 0.0005 |
|---|---|---|---|---|
| | − | − | − | + |
| HA Mw 500 KDa/PHMB | − | − | − | + |
| HA Mw 500 KDa1/PAPB | − | − | − | + |
| PHMB | − | − | −/+ | + |
| PAPB | − | − | + | + |

+ = growth present;
− = growth absent;

### Example 4—Antimicrobial Activity Against Staphylococcus aureus and Staphylococcus epidermidis of Various Soluble Polyelectrolyte Complexes HA/PHMB

The antimicrobial activity of solutions containing 0.1 mEq of PHMB or 1 mEq of PAPB or 1 mEq HA Mw 500 KDa/0.1 mEq PHMB or 1 mEq HA Mw 500 KDa/0.1 mEq PAPB in 10 mL was evaluated.

Biofilm formation assay—Strains of Staphylococcus aureus and Staphylococcus epidermidis were thawed and cultured overnight in Tryptic Soy Broth. The culture broths obtained after overnight incubation were diluted to the concentration of 10⁷ CFU/mL and incubated overnight at 37° C. in 96-well multiwell polystyrene plates with the addition of the substances at the established dilutions. Biofilm formation was assayed by staining the plates with 1% crystal violet and reading the absorbance at 550 nm. No biofilm formation was observed.

Antibacterial activity assay—Culture broths of Staphylococcus aureus and Staphylococcus epidermidis at the concentration of 10⁷ CFU/mL were incubated for 24 h with solutions containing 0.1 mEq of PHMB or 0.1 mEq of PAPB or 1 mEq HA Mw 500 KDa/0.1 mEq PHMB or 1 mEq HA Mw 500 KDa/0.1 mEq PAPB in 10 mL at the dilutions 1:2, 1:5 and 1:10.

Scalar dilutions of the samples thus treated were then plated on Tryptic Soy Agar in order to count the viable CFUs, and incubated for 12 h at 37° C. No bacterial growth was observed for any sample up to the dilution of 1:10.

### Example 5—Antimicrobial Activity Against Staphylococcus epidermidis and Staphylococcus aureus of Hybrid Cooperative Complexes of High- and Low-Molecular-Weight HA with PHMB and PAPB

The hybrid cooperative complex of high- and low-molecular-weight HA was prepared as reported in EP 2 614 090 B 1, using HA 1400 KDa and HA 220 KDa in equivalent amounts.

The two micro-organisms, Staphylococcus epidermidis and Staphylococcus aureus, were cultured for 12 h in TSB medium. The two culture broths were diluted to the concentration of 0.5 OD, and 100 μl was seeded on plates of agarised medium with the aid of a buffer. Holes were made in the seeded plates into which was inserted 100 μL of the following soluble GAG/ABP electrolyte complexes wherein the ratio in equivalents is equal to 10 and wherein the GAG is the hybrid cooperative complex HA 1400 KDa+HA 220 KDa (1:1) and ABP is PHMB in one case and PAPB in the other. Table 4 shows the results obtained.

TABLE 4
Inhibition of the growth of Staphylococcus epidermidis and
Staphylococcus aureus in the presence of soluble GAG/ABP electrolyte
complexes wherein the ratio in equivalents is equal to 10 and wherein
the GAG is the hybrid cooperative complex HA 1400 KDa + HA
220 KDa (1:1) and ABP is PHMB in one case and PAPB in the other.

| | |
|---|---|
| | Zone of inhibition |
| Sample | (cm) |

| Micro-organism | (mEq./10 mL) | Neat | 1:10 |
|---|---|---|---|
| Staphylococcus | Control PHMB | 1.6 | 1.3 |
| epidermidis | HA 1400 KDa + HA | 1.5 | 1.3 |
| | 220 KDa/PHMB | | |
| | Control PAPB | 1.5 | 1.2 |
| | HA 1400 KDa + HA | 1.5 | 1.2 |
| | 220 KDa/PAPB | | |
| Staphylococcus | Control PHMB | 1.4 | 1.0 |
| aureus | HA 1400 KDa + HA | 1.2 | 0.8 |
| | 220 KDa/PHMB | | |
| | Control PAPB | 1.3 | 0.9 |
| | HA 1400 KDa + HA | 1.1 | 0.8 |
| | 220 KDa/PAPB | | |

### Example 6—Wound-Healing Assays on Human Keratinocytes

The biological response of the soluble polyelectrolyte complex of Example 2 was evaluated in wound-healing assays in vitro, using time-lapse videomicroscopy. In particular, a monolayer of immortalised human keratinocytes (HaCat) was scratched to simulate a wound, and the sample to be tested, diluted 1:8, was added to the cells thus damaged. Wound closure was monitored in real time by observation of cell migration and quantitative analysis of repair speed over time. Using suitable software (Okolab), the repair area was evaluated at increasing experimental times for different observation areas (objects) in each plate and for each treatment. The analysis was conducted automatically and manually. Table 5 shows the results in percentage terms of the wound area (scratch) repaired compared with the repair quantified in untreated samples (control), and the repair rate for each sample.

TABLE 5
Wound-healing assays on human keratinocytes. This table shows
the results in percentage terms of the wound area (scratch)
repaired compared with the repair quantified in untreated
samples (control), and the repair rate for each sample.

| | (Repaired area Gel/ |
|---|---|
| | Repaired area |
| Migration rate (mm²/h) | Control) *100 |

| | | |
|---|---|---|
| Time | | mEq GAG/mEq PHMB equal |
| (h) | CTR | to 10. (example 2) |

| | | | |
|---|---|---|---|
| 0-6 | 0.033 ± 0.011 | 0.036 ± 0.002 | 145 ± 10 |
| 6-12 | 0.015 ± 0.008 | 0.016 ± 0.004 | 134 ± 4 |

1. Soluble polyelectrolyte complexes of glycosaminoglycans with biguanide polymer derivatives.

2. Soluble polyelectrolyte complexes according to claim 1, wherein the polymeric biguanidine antimicrobial is polyhexamethylene biguanide or polyaminopropyl biguanide.

3. Soluble polyelectrolyte complexes according to claim 1 wherein the glycosaminoglycans are selected from chondroitin, chondroitin sulphate, hyaluronic acid, complexes and crosslinking products thereof.

4. Soluble polyelectrolyte complexes according to claim 1, wherein the average molecular weight Mn of the polyelectrolytes ranges between 2×10³ and 5×10⁶ Da.

5. Soluble polyelectrolyte complexes according to claim 1, wherein the ratio in equivalents between glycosaminoglycan and polymeric biguanidine antimicrobial is >10.

6. Formulations containing the soluble polyelectrolyte complexes according to claim 1 in combination with excipients and optionally other active ingredients and rheological additives.

7. Formulations according to claim 6 in the form of eyedrops, gel, mouthwash, lotion, ointment or solution.

8. Method of treating acne, infections or inflammations of bladder, vaginal, eye, oral mucosa of a human in need thereof with the formulations according to claim 6, said method comprising
treating said human with a pharmaceutical effective amount of said formulations.
